(19) United States
(12) Patent Application Publication
(10) Pub. No.: US 2020/0185105 A1
(43) Pub. Date: Jun. 11, 2020

(54) MONITORING QUALITY OF PHARMACEUTICAL MANUFACTURING SITES

(71) Applicant: PIRAMAL ENTERPRISES LIMITED, Mumbai (IN)

(72) Inventors: Rashida NAJMI, Mumbai (IN); Monika BORNANI, Mumbai (IN)

(73) Assignee: PIRAMAL ENTERPRISES LIMITED, Mumbai (IN)

(21) Appl. No.: 16/697,387

(22) Filed: Nov. 27, 2019

Related U.S. Application Data
(60) Provisional application No. 62/790,500, filed on Jan. 10, 2019.

(30) Foreign Application Priority Data
Nov. 28, 2018 (IN) 201821044823

(51) Int. Cl. G16H 50/30; G16H 50/20; G16H 40/20; G16H 70/20

(52) U.S. Cl. CPC G16H 50/30; G16H 70/20; G16H 40/20; G16H 50/20

(57) ABSTRACT

The present invention relates to a level meter measuring the quality health of a pharmaceutical manufacturing site and which predicts 24/7 audit readiness for quality outcomes, wherein outcome is interpreted using tangible data; referred to as a Quality Health Barometer. The present invention also relates to a method of evaluating the audit readiness of a pharmaceutical manufacturing site. The present invention further relates to a method of evaluating data integrity (DI) compliance of a pharmaceutical manufacturing site.

### CROSS REFERENCE APPLICATIONS

This application claims the benefit of Indian Provisional Application No.: 201821044823 filed on Nov. 28, 2018 and U.S. Application No. 62/790,500 filed on Jan. 10, 2019.

### FIELD OF THE INVENTION

The present invention relates to a level meter measuring the quality health of a pharmaceutical manufacturing site and which predicts 24/7 audit readiness for quality outcomes, wherein outcome is interpreted using tangible data; referred to as a Quality Health Barometer. The present invention also relates to a method of evaluating the audit readiness of a pharmaceutical manufacturing site. The present invention further relates to a method of evaluating data integrity (DI) compliance of a pharmaceutical manufacturing site.

### BACKGROUND OF THE INVENTION

Pharmaceutical products due to their nature of use need a high level of quality assurance. This is because they are used either as a prophylactic or curative drugs. While being used as a prophylactic they are expected to not lead to any complication or health hazard, whether short term or a long term. While being used as a curative, since they are used on patients who already have an underlying medical condition, the product is expected to cure the condition and while doing so should not cause any adverse reaction.

Unlike other high risk industries like automotive, aerospace which also play with human and animal lives; pharmaceutical industries take a more conservative approach. The reason behind this conservative approach is a high risk due to delayed detection of hazards to the biological race. To explain this, in case of a technical issue with an automobile or an aircraft, while it risks the lives to the extent of hazard when it crashes etc., the risk is immediate and future hazards can be prevented by a recall. In case of pharmaceutical industry, the knowledge of hazard caused by a drug to the human race could surface out after a few years or a decade and by which time there would be no way to reverse the onset of such damage that will definitely occur in those patients who have already consumed that drug. This is the reason pharmaceutical inventions undergo multiple clinical studies before they are commercialized and require a strict adherence to regulatory guidance on a continued basis.

Unfortunately, pharmaceutical regulatory guidance is still under the phase of harmonization and it varies from country to country. For e.g. CFRs in US, EU annexes in Europe, Orange guide in UK, WHO in rest of the world and so on. For a company of a global standard complying to all the regulations where it markets its product individually is an extremely onerous task and complicates its quality system too. This requires a harmonized approach to one quality system that fits to all the norms. In order to do this, it is important for a company to design a common platform to lay its standards, execute and then it is important to understand the stage at which they are in complying with these standards.

“Write what you do and do what you have written” are golden words in GxP compliance. Assessment of these words though is fairly complicated, runs through a stepwise approach in checking around 800 Standard Operating Procedures (SOPs) in a typical Pharma plant, each SOP running into several pages and steps. Most companies use an internal audit program as dip stick to measure the level of compliance. This however is a sample based check and can miss vital over trivial if not performed by an experienced auditor. It also has some level of variable outcome on assessment from auditor to auditor.

Companies also use various metric to understand where they stand in respect of key metrics on compliance. Key metrics are however individually tracked and do not give an overall inference on a company's compliance. What it measures is an intangible sense of compliance. This often lead to a pitfall in compliance focus and surprises during regulatory audit leading to import alert or statement of non-compliance leading to high business and reputation impact as well as non-availability of product to the patients.

Therefore there is a need for a tangible value that can sense compliance risk and obtain the required attention from the quality point of view. It also enables channelizing the attention to where it is needed. Additionally it comes as a ready reckoner to preparing for any regulatory inspections. The inventors of the present invention have provided a quality tool to obtain this quantitative measurement.

### SUMMARY OF THE INVENTION

In one aspect, the present invention relates to a level meter for measuring the quality health of a pharmaceutical manufacturing site and which predicts 24/7 audit readiness for quality outcomes, wherein outcome is interpreted using tangible data; referred to as a Quality Health Barometer (SENSOR™).

In another aspect, the present invention further relates to the level meter, wherein the score on the level meter is the site score for a pharmaceutical manufacturing site under evaluation, which is determined using eleven critical indicators selected from the group consisting of:

1. Audit readiness score card (System Score),

2. Data integrity compliance score,

3. Product Quality Complaints,

4. Invalidated OOS,

5. CAPA closure rate,

6. Process OOS,

7. Investigation Closure Rate,

8. Stability OTIF,

9. Change Control Closure rate,

10. Deviation closure rate and

11. SOP Validity.

In a further aspect, the present invention relates to the level meter, wherein each of the eleven critical indicators is attributed a weightage in percent based on the influence of the indicator on quality as described herein.

In another aspect, the present invention relates to the level meter, wherein each of the eleven critical indicators is allocated a rating between 1 to 5.

In a further aspect, the present invention relates to a method to evaluate the site score as described herein.

In an aspect, the present invention relates to a method of evaluating the ‘Site Health’ for a pharmaceutical manufacturing site under evaluation as described herein.

In another aspect, the present invention relates to a method of evaluating the level of ‘Cure’ required for a pharmaceutical manufacturing site under evaluation as described herein.

In yet another aspect, the present invention relates to a method of evaluating the ‘Risks’ involved and ‘Focus’ required for a pharmaceutical manufacturing site under evaluation as described herein.

In an aspect, the present invention provides a quality health barometer which is a level meter having a scale of 0 to 5 wherein the site obtaining a high score on the level meter qualifies as a Healthy site, which is indicative of an excellent quality culture and corresponds to needed low/medium focus and has low quality risk.

In an aspect, the present invention relates to a quality tool (PREDICT™) used for predicting audit or inspection outcome at pharmaceutical manufacturing sites, wherein measurement is based on intangible data and the outcome is useful to interpret the probable outcome of regulatory inspections at a site.

In another aspect, the present invention provides a method of evaluation (PREDICT™) of the predict system score of a pharmaceutical manufacturing site, wherein the predicted outcome comprises of three elements; first element or audit score (System score) being assessment of GMP system compliance via check points based on 132 vital parameters, second element being SME readiness, which integrates aspects of SME readiness at pharmaceutical manufacturing sites and the third element being data integrity compliance scores of pharmaceutical manufacturing site.

In another aspect, the present invention provides the method, wherein the 132 parameters of the first element are categorized into three levels namely Patient Risk (Level-I), Product Quality Risk (Level-II) and Compliance Risk (Level-III).

In another aspect, the present invention provides a method of assessing the first element or audit score as described herein.

In yet another aspect, the present invention provides a method of assessing the second element or the SME readiness as described herein.

In a further aspect, the present invention provides a method of assessing the third element or data integrity compliance by providing a score in percentage (%) based on evaluating compliance level at the site with regards to regulatory requirements (163 parameters) and also level of automation (Mainly in Quality control. Quality Assurance and Production department) at each pharmaceutical manufacturing sites.

In another aspect, the present invention provides a method of evaluating site score of a pharmaceutical manufacturing site, wherein the predicted outcome is determined by compilation of GMP audit score(system score), SME score and data integrity compliance score of the site.

In one aspect, the present invention provides a quality tool (CALCULUS™) used for evaluating the data integrity compliance at pharmaceutical manufacturing sites, wherein measurement is based on quantified data and the outcome is useful to interpret the probable outcome of regulatory inspections at a site.

In another aspect, the present invention provides a method of evaluation (CALCULUS™) of the data integrity compliance at a pharmaceutical manufacturing site which is determined based on regulatory requirements and also internal need for increasing and elevating automation to reduce possibility of errors.

In another aspect, the present invention provides the method, wherein the data integrity compliance is provided a score (DI score) computed by considering the sum of a weightage of 85% for a first component including a data integrity checklist score and 15% for a second component including level of automation at site.

In yet another aspect, the present invention provides the method, wherein the first component for measuring the data integrity compliance is based on data integrity checklist consisting of 163 parameters for assessing compliance level of sites with regards to regulatory requirements as described herein.

In a further aspect, the present invention provides the method, wherein the second component for measuring the data integrity compliance is based on the level of automation at site comprising weightage of 5% for Quality Control, 5% for Quality Assurance and 5% for Manufacturing.

In an aspect, the present invention provides the method, wherein the data integrity compliance at pharmaceutical manufacturing site requires a threshold value of >70%.

In an aspect, the pharmaceutical manufacturing site is provided a rating based on the DI score as described herein.

These and other aspects and advantages of the present invention will be apparent to those skilled in the art from the following description.

### BRIEF DESCRIPTION OF DRAWINGS OF THE INVENTION

FIG. 1 represents a schematic functioning of Quality Health Barometer (SENSOR™).

FIG. 2 represents a Quality Health Barometer with a rating of 0 to 5.

FIG. 2A represents the baseline score of “Site A” on Quality Health Barometer.

FIG. 2B represents the improvised score of “Site A” on Quality Health Barometer.

FIG. 3A represents the baseline score of “Site B” on Quality Health Barometer.

FIG. 3B represents the improvised score of “Site B” on Quality Health Barometer.

FIG. 4 represents a schematic representation of Predict™ Outcome Model.

FIG. 5 represents a schematic representation for system score (Audit score) (First element)

FIG. 6 represents Predict™ Interpretation and Outcome

FIG. 7 represents a schematic process flow representation of Audit score (System Score)

FIG. 8 represents Predict™ Outcome of Example 3

FIG. 9 represents Predict™ Outcome of Example 4

FIG. 10 represents Predict™ Outcome of Example 5

FIG. 11 represents Predict™ Outcome of Example 6

FIG. 12 represents a schematic representation of qualifying parameters for Data integrity (CALCULUS™)

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the description herein, may utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs.

### Definitions

For the purpose of the disclosure, listed below are definitions of various terms used to describe the present invention. Unless otherwise indicated, these definitions apply to the terms as they are used throughout the specification and the appended claims, either individually or as part of a larger group. They should not be interpreted in the literal sense. They are not general definitions and are relevant only for this application.

It should be noted that, as used in this specification and the appended claims, the singular forms “a,” “an,” and “the” include plural referents unless the content clearly dictates otherwise.

It should be noted that the term “or” is generally employed in its sense including “and/or” unless the content clearly dictates otherwise.

Audit score card (System score) provides a score evaluating the audit readiness of a pharmaceutical manufacturing site encompassing of three elements; first element being assessment of GMP system compliance via check points based on 132 vital parameters, second element being SME readiness, which integrates aspects of SME readiness at pharmaceutical manufacturing sites and the third element being data integrity compliance scores of pharmaceutical manufacturing site.

Data integrity compliance score provides an evaluation of the data integrity (DI) compliance of a pharmaceutical manufacturing site which is determined based on regulatory requirements and also internal need for increasing and elevating automation to reduce possibility of errors and this measurement includes assessing compliance level of sites with regards to regulatory requirements (163 parameters) and also level of automation (Mainly in Quality control, Quality Assurance and Production department) at each pharmaceutical manufacturing sites.

Product Quality Complaint refers to a number of complaint involving any possible, including actual, failure of a drug to meet any of its specifications designed to ensure that any drug conforms to appropriate standards of identity strength, quality, and purity. Complaints severity is usually monitored as number of Critical, Major or Minor complaints.

The term “Out Of Specification” (OOS) results includes all test results that fall outside the specifications or acceptance criteria established in drug applications, drug master files (DMFs), official compendia, or by the manufacturer. The term also applies to all in-process laboratory tests that are outside of established specifications.

The term “Invalidated OOS” refers to total OOS test results for lot release and long-term stability testing invalidated by the covered establishment due to an aberration of the measurement process divided by the total number of lot release and long-term stability tests performed in the same current reporting period.

The term “Corrective Action Preventive Action” (CAPA) refers to a systematic approach that includes actions needed to correct (“correction”), prevent recurrence (“corrective action”), and eliminate the cause of potential nonconforming product and other quality problems (preventive action). It involves collecting information, analyzing information, identifying and investigating product and quality problems, and taking appropriate and effective corrective and/or preventive action to prevent their recurrence. CAPA closure rate determines the percentage of on-time closure of an issue at the pharmaceutical manufacturing site.

FDA regulations require that an investigation be conducted whenever an OOS, lab incidence, complaints, deviation, etc. occurs. The purpose of the investigation is to determine the cause of the event. Even if a batch is rejected based on an OOS, deviation, complaints, etc. the investigation is necessary to determine if the result is associated with other batches of the same drug product or other products. The investigation should be thorough, timely, unbiased, well-documented, and scientifically sound. Investigation closure rate determines the percentage of on-time closure of an investigation at the pharmaceutical manufacturing site.

Stability OTIF is calculated as the percentage of completion of stability studies for a set of pharmaceutical samples in a given time.

Change control is a CGMP concept that focuses on managing change to prevent unintended consequences. Certain manufacturing changes (i.e. changes that alter specifications, a critical product attribute or bioavailability) require regulatory filings and prior regulatory approval. Change is an inherent part of the life cycle of a pharmaceutical product. A change can be an addition to, deletion of, or modification to manufacturing facility, utilities, process, material, product, procedures or equipment (including software) which impacts quality or regulatory requirements. A change control system provides checks and balances in the quality system by tracking, reviewing and approving the changes. In adequate change control procedures ends up in regulatory non-compliance. The purpose of change control is to prevent the unintended consequences that are sometimes encountered when making a change to a product or system. Change control closure rate determines the percentage of on-time closure of a change control at the pharmaceutical manufacturing site.

A deviation is a departure from an approved procedure or established standard or specification. A deviation which occurs in unplanned manner due to system failure or equipment breakdown or manual error shall be termed as an unplanned deviation. Deviations should be investigated to ascertain whether they could have an impact on the product quality. Deviation closure rate determines the percentage of on-time closure of a deviation at the pharmaceutical manufacturing site.

For Standard Operating Procedures (SOP), periodic revision of SOP within stipulated timeline are measured and scored accordingly.

In an embodiment, there is provided a level meter measuring the quality health of a pharmaceutical manufacturing site and which predicts 24/7 audit readiness for quality outcomes, wherein outcome is interpreted using tangible data; referred to as a Quality Health Barometer.

In a further embodiment, there is provided a quality health barometer which is a level meter having a scale of 0 to 5 wherein the site obtaining a high score on the level meter qualifies as a Healthy site, which is indicative of an excellent quality culture and corresponds to needed low/medium focus and has low quality risk. This provides a comfort to the organization that such sites are not in the radar for any regulatory action leading to business derailment and growth impact.

The term “Quality metrics” means all those key indicators that give an idea of the collective quality characteristics of a certain manufacturing site. These indicators are quantitative and represent data in numerical form and thereby identify quality culture.

In an embodiment, there is provided a method of determining the score on the barometer using eleven defined critical indicators.

In a further embodiment, the critical indicators are selected from the group consisting of:
1. Audit score card (System Score)
2. Data integrity compliance score
3. Product Quality Complaints
4. Invalidated OOS
5. CAPA closure rate
6. Process OOS
7. Investigation Closure Rate
8. Stability OTIF
9. Change Control Closure rate
10. Deviation closure rate
11. SOP Validity

In an embodiment Audit score card (System score) is considered for scoring. Audit Readiness checklist (System Score) method is used to evaluate the audit readiness of a pharmaceutical manufacturing site via an Audit score card encompassing within itself some of the key check points, based on 132 vital parameters with varying criticality based on patient risk, product risk and pharmaceutical GMP risk linked to FDA's six systems. Based on compliance level at sites, scores are assigned to each of 132 parameters and final scoring is done and scores are considered as a part of SENSOR™ final scores computation.

In an embodiment, data integrity compliance scores are considered for scoring. Scores obtained from evaluating data integrity checklist and automation level at pharmaceutical manufacturing site are further integrated and considered for SENSOR final scoring.

In an embodiment, for SENSOR™ score there is evaluation on number of severity of complaints received and sites are evaluated on site specific level targets for reduction of complaints and are constantly monitored. The site score is constantly improved by revalidating the threshold value to a better level of compliance.

In another embodiment, there is evaluation on an invalidated OOS rate to determine percentage of number of invalidated OOS verses total number of test performed for lot release and long-term stability in the reporting period. The site score is constantly improved by revalidating the threshold value to a better level of compliance.

In an embodiment, there is provided a CAPA closure rate to determine the percentage of on-time closure of an issue at the pharmaceutical manufacturing site. Evaluation is done based on timely closure of identified CAPAs. The site score is constantly improved by revalidating the threshold value to a better level of compliance.

In an embodiment, there is provided an indicator to identify the number of out of specification (Process OOS) batches over the total number of batches manufactured in a given period. The site score is constantly improved by revalidating the threshold value to a better level of compliance.

In an embodiment, there is provided a number of investigations closed over the number of investigation due for closure in a given period. The site score is constantly improved by revalidating the threshold value to a better level of compliance.

In an embodiment, with respect to Stability OTIF, there is provided the number of samples analyzed over number of samples due for analysis. The site score is constantly improved by revalidating the threshold value to a better level of compliance.

In an embodiment, there is provided on-time closure of Change Controls over the total number of Change controls due for closure in a given period. The site score is constantly improved by revalidating the threshold value to a better level of compliance.

In an embodiment, there is provided on-time closure of Deviations over the total number of Deviations due for closure in a given period. The site score is constantly improved by revalidating the threshold value to a better level of compliance.

In an embodiment, with respect to time revision of Standard Operating Procedures (SOPs) over the ones which are due for revision in a given period. The site score is constantly improved by revalidating the threshold value to a better level of compliance.

In an embodiment, each of the eleven critical indicators defined herein is attributed a weightage in percent based on the influence of the indicator on quality.

In an embodiment, each of the eleven critical indicators is attributed a weightage in percent based on the influence of the indicator on quality, selected from:
(a) Audit score card (System score) with a weightage of 22%,
(b) Data integrity compliance, complaints, Invalidated OOS, CAPA closure rate, Investigation closure rate, Stability OTIF, Change control closure rate & Deviation closure rate with a weightage of 9% each and
(c) Process OOS and SOP validity with a weightage of 3% each.

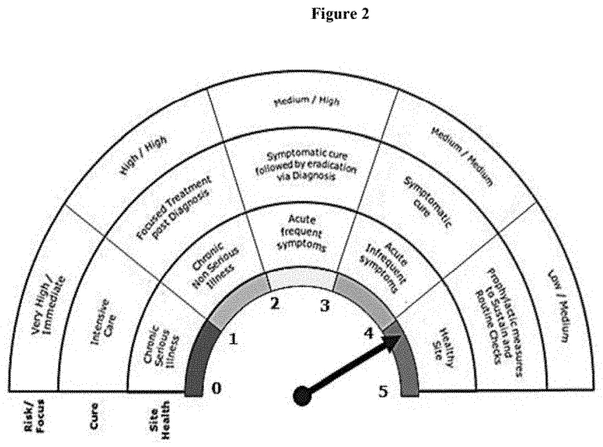

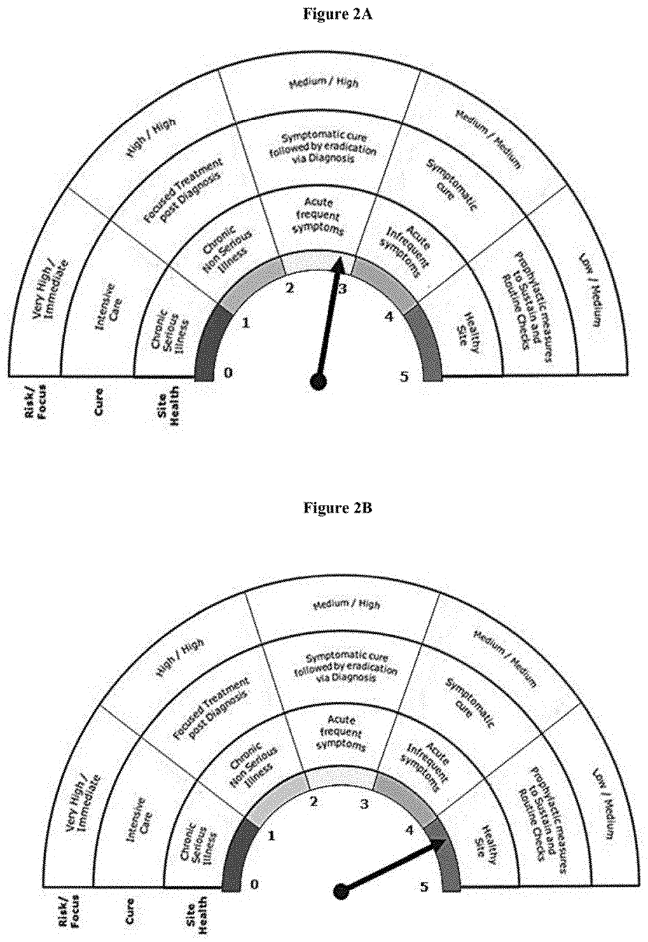

In an embodiment, each of the eleven critical indicators defined herein is allocated a rating between 1 to 5 which are depicted in FIG. 2.

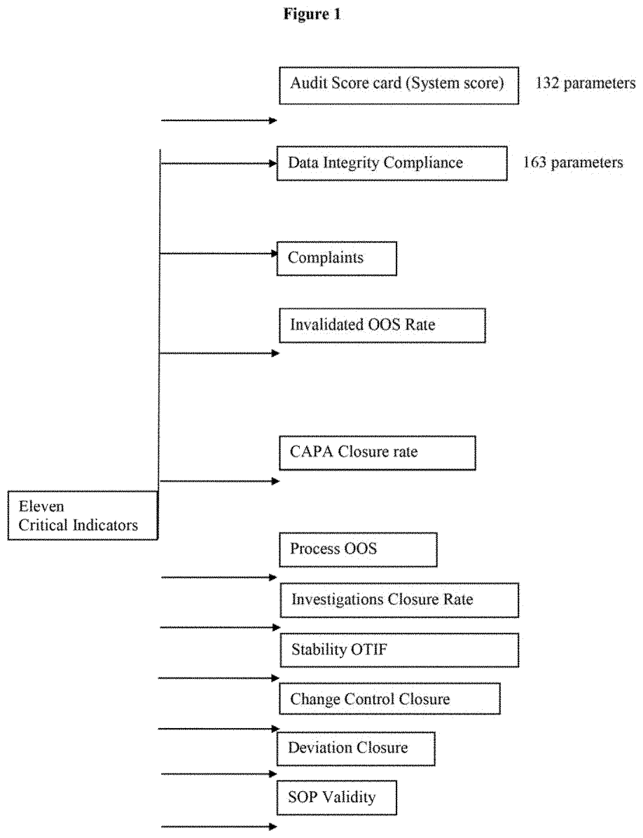

In an embodiment, there is provided a method to evaluate the site score, comprising the steps of:
(a) providing audit score card (System score) with a weightage of 22%;
(b) providing data integrity compliance, complaints, Invalidated OOS, CAPA closure rate, Investigation closure rate, Stability OTIF, Change control closure rate & Deviation closure rate with a weightage of 9% each;
(c) providing process OOS and SOP validity with a weightage of 3% each;
(d) providing ratings on scale of 1 to 5 based on threshold value and targets for improvements assigned, and their current status, wherein:
   ‘5’ being considered as 100% achievement over the target assigned and
   ‘1’ being considered as achievement way below minimum expected level of target;
(e) obtaining the score for each of the eleven critical indicators as a product of weightage of steps (a) to (c) and the rating of step (d);
(f) obtaining the site score by adding the scores of each of the eleven critical indicators of step (e) and
(g) mapping the site score obtained in step (f) onto the level meter with a pointer configured by application of software to indicate a region on the level meter in order to gauge the performance of the site.
   FIG. 1 depicts schematic functioning of SENSOR™.

In a further embodiment, the level meter is demarcated into 5 colored regions corresponding to the score and indicative of Site Health, selected from:
a) Score of 0-1 corresponding to red region indicating that the Site Health has Chronic Serious Illness,
b) Score of 1-2 corresponding to orange region indicating that the Site Health has Chronic Non-serious Illness,
c) Score of 2-3 corresponding to yellow region indicating that the Site Health has Acute Frequent Symptoms,
d) Score of 3-4 corresponding to light green region indicating that the Site Health has Acute In-frequent Symptoms, and
e) Score of 4-5 corresponding to green region indicating that the Site is Healthy.

In another embodiment, the level meter is demarcated into 5 colored regions corresponding to the score and indicative of Cure, selected from:
a) Score of 0-1 corresponding to red region indicating that the Cure required is Intensive Care,
b) Score of 1-2 corresponding to orange region indicating that the Cure required is Focused Treatment post Diagnosis,
c) Score of 2-3 corresponding to yellow region indicating that the Cure required is Symptomatic cure followed by eradication via Diagnosis,
d) Score of 3-4 corresponding to light green region indicating that the Cure required is Symptomatic cure, and
e) Score of 4-5 corresponding to green region indicating that the Cure required is Prophylactic measures to Sustain and Routine Checks.

In yet another embodiment, the level meter is demarcated into 5 colored regions corresponding to the score and indicative of Risks and Focus, selected from:
a) Score of 0-1 corresponding to red region indicating that the Risk is Very High and Focus is Immediate,
b) Score of 1-2 corresponding to orange region indicating that the Risk is High and Focus is High,
c) Score of 2-3 corresponding to yellow region indicating that the Risk is Medium and Focus is High,
d) Score of 3-4 corresponding to light green region indicating that the Risk is Medium and Focus is Medium, and
e) Score of 4-5 corresponding to green region indicating that the Risk is Low and Focus is Medium.

In an embodiment, there is provided a quality tool (PREDICT™) used for predicting audit/inspection outcome at pharmaceutical manufacturing sites, wherein measurement is based on intangible data and the outcome is useful to interpret the probable outcome of regulatory inspections at a pharmaceutical manufacturing site.

In an embodiment, there is provided a method of evaluation (PREDICT™) of the predict system score of a pharmaceutical manufacturing site, wherein the predicted outcome comprises of three elements:
1. The first element or audit score assesses the GMP six system compliance via check points. These check points are based on 132 vital parameters with varying criticality based on patient risk, product risk and pharmaceutical GMP risk linked to FDA's six systems.
2. The second element is SME readiness. It further integrates aspects of SME readiness at sites which is key for successful audit outcome.
3. The third element is data integrity compliance scores as data integrity (DI) forms integral part of good quality system.

All three elements together form basis of predicting audit outcome. Predict™ outcome is combination of tangible and intangible data providing visibility to management on what would be outcome if audit/inspection is triggered at sites.

In an embodiment, the six systems of the first element are selected from the group consisting of:
(i) Facility And Equipment System,
(ii) Laboratory Control System,
(iii) Material System,
(iv) Packaging and Labelling System,
(v) Production System, and
(vi) Quality Management Systems.

In an embodiment, the Facility And Equipment System comprises of the parameters selected from the group consisting of:
1. Equipment cleaning and sanitization,
2. Prevention of cross-contamination, isolation and containment,
3. Air handling systems,
4. Appropriate use of equipment operations substances,
5. Calibration program,
6. Clean rooms control, maintenance and cleaning,
7. Control system for implementing changes in the equipment,
8. Documented investigation into any unexpected discrepancy,
9. Environmental zoning,
10. Equipment surfaces should not be reactive, additive, or absorptive,
11. Facilities Maintenance,
12. Installation Qualification/Operational Qualification/Performance Qualification (IQ/OQ/PQ) of facilities, utilities and equipment,
13. Informational Technology Good Manufacturing Practices (IT GMP), validation and security,
14. Planned preventive maintenance program,
15. Adequacy of equipment design, size, and location,
16. Design guidelines,
17. Equipment identification practices,
18. Facilities Cleaning,
19. Layout & drawings,
20. Lighting, potable water, washing and toilet facilities, sewage and refuse disposal,
21. Pest control,
22. Sanitation of the building,
23. Technical/Design files, and
24. Zone Ownership.

In an embodiment, the Laboratory Control System comprises of the parameters selected from the group consisting of:
1. Laboratory methods, standards and controls,
2. Qualification and Validation of all QC methods,
3. Qualification of all Laboratory Equipment,
4. Raw data definition, standards, control and verification,
5. Reference standards management,
6. Retained samples, requirements and management,
7. Specifications, standards, and sampling plans,
8. Stability testing,
9. Acceptance Activities and in-process release,
10. Adequacy of equipment and facility for intended use,
11. OOS & Out of trend (OOT) Investigations,
12. Sample handling, storage, and integrity,
13. Test data review and authorization,
14. Validation and security of computerized or automated processes,
15. Adequacy of staffing for laboratory operations,
16. Calibration program,
17. Equipment List,
18. Maintenance program,
19. Method validation policy and standards, and
20. Trending, reporting, and statistical quality control.

In an embodiment, the Material System comprises of the parameters selected from the group consisting of:
1. Bill of Materials,
2. Environmentally controlled storage conditions,
3. Finished product distribution records by lot,
4. Identification & Quality Status,
5. Lot traceability of components making up a batch,
6. Purified Water system control,
7. Specifications and acceptance testing of raw materials,
8. API, excipient, reagent and reference material control,
9. Control of quarantine goods,
10. Control of reject goods,
11. Expiry dating and retest requirements,
12. Incoming goods acceptance checks,
13. Lot numbering control,
14. Pack Range Control—Artwork, components, pack codes,
15. Qualification of cold chain or supply chain,
16. Reconciliation,
17. Reprocessing/rework control,
18. Sampling plan,
19. Supplier management,
20. Authority to Destroy and destruction of records,
21. Control of Distributors, records, Technical Agreements,
22. Control of returned or salvaged goods,
23. Inventory Management,
24. Raw materials segregation and labeling,
25. Testing or validation of supplier's test results for components, containers and closures, and
26. Warehouse controls and First-in-first-out (FIFO).

In an embodiment, the Packaging and Labelling System comprises of the parameters selected from the group consisting of:
1. Adequate inspection (proofing) of incoming labeling,
2. Control of bulk and unlabelled product,
3. Examination of the labeled finished product,
4. In-process inspection of product,
5. Master Packaging Instructions and Records,
6. QA In-process control checks for labeling/packaging operations,
7. Specifications for packaging & labeling materials,
8. Conformance to tamper-evident packaging (TEP) requirements,
9. Controls and management of packaging operations,
10. Line clearance, inspection, and documentation,
11. Product segregation and labeling,
12. Sampling plan and acceptance operations for packaging and labeling materials,
13. Storage, issue, inspection and reconciliation of labels and printed materials, returns after issue,
14. Validation and security of computerized/automated labeling/packaging processes,
15. Validation of packaging and labeling operation,
16. Control of issuance of labeling, examination of issued labels and reconciliation of used labels,
17. Monitoring of printing devices, and
18. Physical/spatial separation between different labeling and packaging lines.

In an embodiment, the Production System comprises of the parameters selected from the group consisting of:
1. Component cleaning validation,
2. Dispensary operations,
3. Equipment cleaning & use logs,
4. In process controls,
5. In-process and final product specifications,
6. Justification and consistency of in-process specifications and drug product final specifications,
7. Key manufacturing processes,
8. Master Manufacturing Instructions and Records,
9. Process validation,
10. Process validation (including CSV and security of computerized/automated processes),
11. Validation of homogeneity.
12. Validation of shelf life (stability testing),
13. Adequate procedure and practice for charge-in of components,
14. Development products manufacture and controls (Technology Transfer),
15. Environmental monitoring,
16. Gowning regimes and requirements,
17. Personnel entry qualification,
18. Personnel hygiene & medical fitness,
19. Pre-process checks, line clearance, and equipment cleaning,
20. Yield calculations and acceptance limits at critical process stages,
21. Control of microbiological spoilage,
22. Facility cleaning validation,
23. Identity of equipment contents, phase of manufacture/status, and
24. Process descriptions (by process).

In an embodiment, the Quality Management Systems comprises of the parameters selected from the group consisting of:
1. Adverse Drug Event (ADE) Management,
2. Annual Product Review, controls charts and summary,
3. Batch manufacturing record/Batch packing record (BMR/BPR) reviews, approval, archival and retrieval,
4. Customer complaint management,
5. Management of CAPA (FAR, FAR closure and concomitant CAPA),
6. Non-conforming materials, root cause investigation and impact assessment,
7. Previous regulatory observation closure, Establishment Inspection Report (EIR) and Regulatory compliance,
8. Recall Management,
9. Risk assessment and Mitigation Plans,
10. Stability programme management,
11. Technology transfer,
12. Validation management including Validation master plan (VMP) and Quality peer review,
13. Change control (In plant modification, material handling, packaging & labelling etc.),
14. Deviation Management (product, process & utilities),
15. Product disposition,
16. Quality audits and auditing,
17. Documentation management, Record management and Archive—check for data traceability and real time recordings, alignment of e-copies and respective hard copies generated.
18. Management Review and Escalation procedure,
19. Quality Planning, and
20. Training & Qualification Management.

In an embodiment, each of the parameters is categorized into three levels namely Patient Risk (Level-I), Product Quality Risk (Level-II) and Compliance Risk (Level-III). For instance, the parameter “Training & Qualification Management” is categorized into Compliance Risk level while the parameter “Product segregation and labeling” is categorized into Product Quality Risk level” and “Prevention of cross-contamination, isolation and containment” is categorized into Patient Risk.

In an embodiment, there is provided a method to evaluate the audit readiness of a pharmaceutical manufacturing site via an Audit score card (System score) comprising quantification of each of the 132 parameters, based on level of compliance, each of the 132 parameters are assigned with ratings on a scale of 0 to 4 wherein:
0 signifies “No process available”,
1 signifies “Non-compliant process”,
2 signifies “Partially compliant process”,
3 signifies “Compliant with improvements” and,
4 signifies “Highly compliant process”.

As illustrations:
All parameters which are classified as Level-I can be assigned a scoring of ‘0’, ‘3’ or ‘4’ based on the level of compliance as defined herein above.
All parameters which are classified as Level-II can be assigned a scoring of ‘0’, ‘2’, ‘3’ or ‘4’ based on the level of compliance as defined herein above.
All parameters which are classified as Level-III can be assigned a scoring of ‘0’, ‘1’, ‘2’, ‘3’ or ‘4’, based on the level of compliance as defined herein above.

In an embodiment, each of the 132 parameters are associated with a weightage (%) based on its relevance to patient (highest weightage), product and compliance; providing a rating (from 0 to 4) to each of the parameters and obtaining the audit score for each of the parameters as a product of weightage and the rating. The final audit score would be an average of the individual scores.

Considering the factors indicated above, each of the manufacturing site will perform their assessment and provide ratings based on the level of compliance, the scores will then be validated during corporate audits. Scores computed by corporate auditor will be considered as final and resultant score would be on a scale of 1 to 24. The determination of site being audit compliant is based on the scores wherein:
(i) Scores ranging from above 22-24 indicates that the site is highly compliant.
(ii) Scores ranging from above 18-22 indicates that the site is compliant with scope of improvement;
(iii) Scores ranging from 14-18 indicates that the site is partially compliant with gaps; and
(iv) Scores less than 14 indicates that the site is non complaint.
(v) Scoring is done on scale of 1 to 24 and score is interpreted as depicted in FIG. 5.

In an embodiment, there is provided a method, wherein the first element is assessed according to the steps comprising of:
(i) associating each of the 132 parameters with a weightage (%) based on its relevance to patient (highest weightage), product and compliance;
(ii) providing a rating to each of the parameters;
(iii) obtaining the audit score for each of the parameters as a product of weightage and the rating; and
(iv) obtaining the final audit score on a scale of 1 to 24 as an average of the individual scores wherein:
   a) Scores ranging from above 22-24 indicates that the site is highly compliant,
   b) Scores ranging from above 18-22 indicates that the site is compliant with scope of improvement;
   c) Scores ranging from 14-18 indicates that the site is partially compliant with gaps; and
   d) Scores less than 14 indicates that the site is non complaint.

In an embodiment, the second element provides for Subject Matter Expert (SME), evaluated and scored by corporate auditors based on the performance of site during corporate audit. The scoring criteria for SME level are defined as follows:—
“0” Need major change in SME at all levels including quality
“1” Few critical SMEs (audit facing) in quality requires replacement.
“2” Few critical SME (audit facing) within other 5 systems requires replacement
“3” Challenge only with 1 or 2 SME (audit facing) which can be bridged by other competent staff under or at Peer level.
“4” Gap in Quality SME (audit facing) but can be trained
“5” Gap in few other SMEs (audit facing) but can be trained
“6” SME (mainly Quality) require extensive level of audit facing training while possessing requisite knowledge
“7” SME (mainly but not only quality) require major level of strategic audit facing training.
“8” SME (mainly but not only quality) require low level of audit facing training.
“9” SME (mainly quality) requires only incidence related direction or guidance during audit to defend our case as required by regulation during inspection when required.
“10” SME (mainly quality) is self-sufficient in changing the course of inspection in our favour due to strong regulatory knowledge and negotiation capability.

In an embodiment, the third element provides for scores from Data Integrity (DI) compliance and provides percentage compliance level at each site with regards to regulatory requirements (163 parameters) and also level of automation (Mainly in Quality control, Quality Assurance and Production department) at each pharmaceutical manufacturing sites.

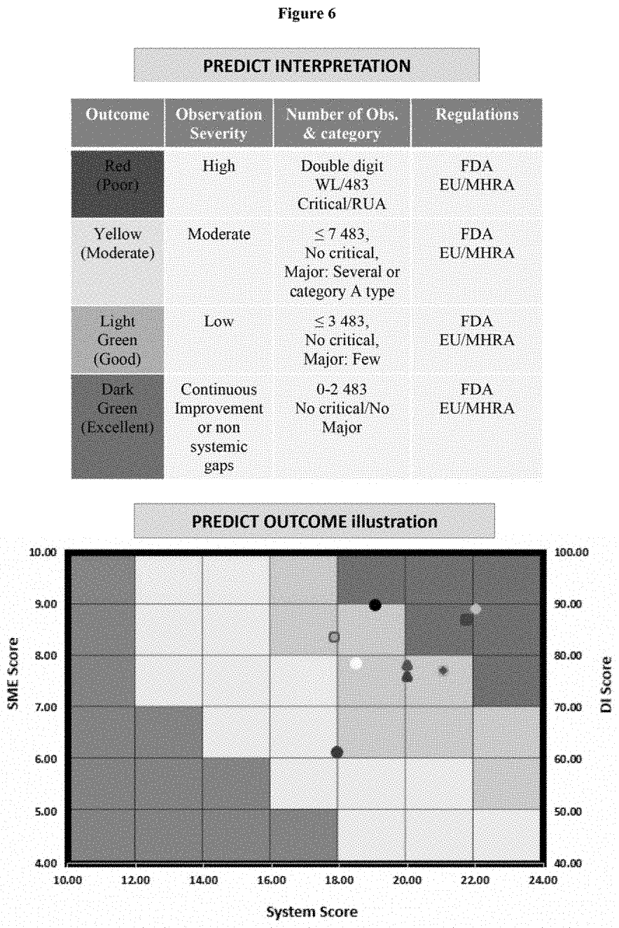

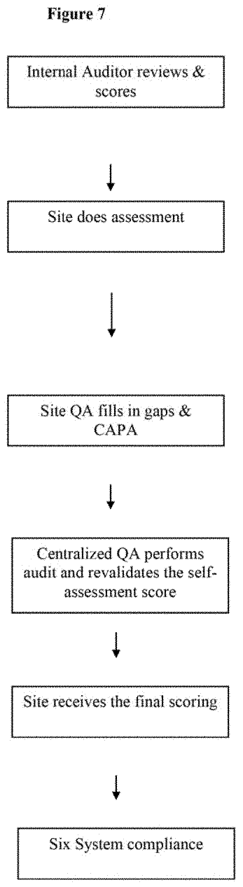

In a further embodiment the predicted outcome is determined by compilation of Audit score (system score), SME score & DI score of respective site. Interpretation of sites outcome and scores is depicted in FIG. 6.

Based on compilation of the scores in FIG. 6, predict outcome is interpreted as provided in Table 1.

TABLE 1
Predict ™ Interpretation

| | Predict | | | |
|---|---|---|---|---|
| Predict | System | Observation | Number & | |
| Outcome | Score | Severity | category | Regulations |
| Red (Poor ) | 1 | High | Doable | FDA |
| | | | digit | EU/MHRA |
| | | | WL/483 | |
| | | | Critical/RUA | |
| Yellow | 3 | Moderate | ≤7 483, | FDA |
| (Moderate) | | | No critical, | EU/MHRA |
| | | | Major: | |
| | | | Several or | |
| | | | category A | |
| | | | type | |
| Light | 4 | Low | ≤3 483, | FDA |
| Green | | | No critical, | EU/MHRA |
| (Good) | | | Major: Few | |
| Dark | 5 | Continuous | 0-2 483 | FDA |
| Green | | Improvement | No | EU/MHRA |
| (Excellent) | | or non- | critical/No | |
| | | systemic gaps | Major | |

In an embodiment, there is provided a quality tool (CALCULUS™) used for evaluation of the data integrity compliance at pharmaceutical manufacturing sites, wherein measurement is based on intangible data and the outcome is useful to interpret the probable outcome of regulatory inspections at a site.

In an embodiment, there is provided a method of evaluation (CALCULUS™) of the data integrity compliance at a pharmaceutical manufacturing site, which is determined based on regulatory requirements and also internal need for increasing and elevating automation to reduce possibility of errors.

In an embodiment, there is provided a first component for data integrity compliance measurement, which includes assessing compliance level of sites with regards to regulatory requirements (163 parameters) and second component including level of automation at each site.

In an embodiment, the parameters used to evaluate the data integrity compliance of a pharmaceutical manufacturing site are selected from the categories as under:
(a) Recording and collection of data
(b) Original record/True copy
(c) Excluding data,
(d) Data Processing
(e) Data transfer/mitigation
(f) Data Governance
(g) Data Integrity Risk Assessment (DIRA)
(h) Computerized System transactions
(i) Computerized system User access/System Administrator role
(j) Audit Trail
(k) Electronic Signatures
(l) Data review and approval
(m) Data Retention
(n) Back up and Archive
(o) File structure
(p) Validation
(q) IT supplier and Service provider
(r) Quality Management Systems (QMS)
(s) Calibration
(t) Quality Control
(u) Standalone Systems
(v) Trainings
(w) Manufacturing

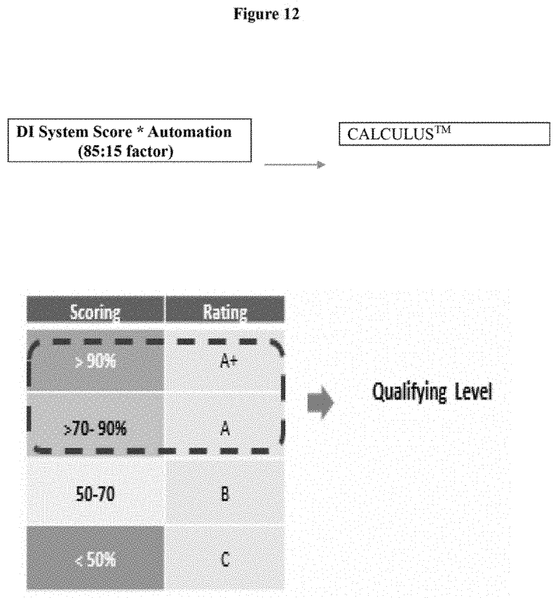

In an embodiment, the data integrity compliance is based on the regulatory (FDA, MHRA, PIC/S etc.) guidance on Data Integrity and is evaluated based on the accuracy, reliability and consistency of the system, process and content. The threshold value for qualifying as data integrity compliant site is >70% as depicted in FIG. 12.

Data Integrity score are computed by considering below weightage of score as follows:—

DI Score=Data integrity checklist score (85%)+Level of automation at site (15%)

Level of automation at site is computed as per criteria set out in Table 2.

TABLE 2
Level of automation scoring criteria (15%)

| Quality Control (5%) | Quality Assurance (5%) | Manutacturing (5%) |
|---|---|---|
| Automation within Quality | Automation within Quality | Automation within |
| Control labs (HPLC and other | Assurance (Document | Manufacturing (shop |
| instruments) are considered | management system, training | floor) is considered |
| and compliance percentage is | system, aberration handling | (Having PLC, SCADA |
| allotted | systems etc.) are considered and | etc.) are considered |
| | compliance percentage is allotted | and compliance |
| | | percentage is allotted |

Note:-
Percentage may vary based on assessment
PLC: Programmable Logic Controller
SCADA: Supervisory Control and Data Acquisition

Modality of Implementation:
The checklist is designed by the Central Quality team based on current regulatory requirements and provides them to the sites.
Site team does gap assessment based on checklist.
Central Quality team evaluates and provides scoring.
Further extent of automation at each site is evaluated by Central quality team and scores are computed.
Sites falling below qualifying level are given immediate attention for remediation
Site team works on gaps and improves the scores.

In an embodiment, the pharmaceutical manufacturing site is provided a rating based on the DI score as follows:
(i) the rating of A+ is assigned to a site with DI score of >90%;
(ii) the rating of A is assigned to a site with DI score of >70-90%;
(iii) the rating of B is assigned to a site with DI score of 50-70%;
(iv) the rating of C is assigned to a site with DI score of <50%; and
(v) the qualifying rating for the site as data integrity compliant is A+ or A (as described in FIG. 12).

The present invention will be more readily understood by referring to the following examples which are given to illustrate the invention but do not limit its scope.

### EXAMPLES

### Example 1

Implementation of Quality Health Barometer at Site A:

The baseline score of “Site A” prior to implementation of Quality Health Barometer was determined to be 2.9 which means the overall site health is considered to have “Acute frequent symptoms of quality issue” and needs improvement.

Using the Quality Health Barometer, eleven critical indicators of quality were evaluated for “Site A”. The score is tabulated in Table 3:

TABLE 3
“SITE A” - Baseline score

| | | | | Overall Score |
|---|---|---|---|---|
| | | | | 2.9 |
| | | | | Overall |
| | | | | Site Health |
| | | | | Needs |
| | | Weight- | | improvement |
| Sr. No. | Indicator | age | Rating | Score |
| 1 | Audit Score card | 22.0% | 4 | 0.88 |
| 2 | Data Integrity Compliance | 9.0% | 4 | 0.36 |
| 3 | Complaint rate | 9.0% | 4 | 0.36 |
| 4 | Invalidated OOS | 9.0% | 5 | 0.45 |
| 5 | CAPA Closure rate | 9.0% | 1 | 0.09 |
| 6 | Process OOS | 3.0% | 5 | 0.15 |
| 7 | Investigations Closure | 9.0% | 3 | 0.27 |
| 8 | Stability OTIF | 9.0% | 1 | 0.09 |
| 9 | Change Control Closure | 9.0% | 1 | 0.09 |
| 10 | Deviation Closure | 9.0% | 1 | 0.09 |
| eleven | SOP Validity | 3.0% | 3 | 0.09 |

From Table 3 it was observed that, there was a need to improve the score on many of the indicators namely CAPA closure rate, Investigation closure, Stability OTIF, Change control closure, Deviation Closure, SOP validity as depicted in FIG. 2A to improve the health of “Site A”. Accordingly, measures were taken to improvise on all critical indicators with continuous monitoring of sustenance of other indicators so as to bring the site score to 4.3 which terms Site A as a “Healthy Site”. The improvised scores are depicted in table 4. In other words, the risk at said site is now considered to be Low and the focus on quality issue is Medium with Prophylactic measures to sustain routine checks. The result on the quality health barometer is depicted in FIG. 2B.

TABLE 4
“SITE A” - Improvised score

| | | | | Overall Score |
|---|---|---|---|---|
| | | | | 4.3 |
| | | | | Overall |
| | | | | Site Health |
| | | Weight- | | Very Healthy |
| Sr. No. | Indicator | age | Rating | Score |
| 1 | Audit Score card | 22.0% | 4 | 0.88 |
| 2 | Data Integrity Compliance | 9.0% | 4 | 0.36 |
| 3 | Complaint rate | 9.0% | 4 | 0.36 |
| 4 | Invalidated OOS | 9.0% | 5 | 0.45 |
| 5 | CAPA Closure rate | 9.0% | 5 | 0.45 |
| 6 | Process OOS | 3.0% | 5 | 0.15 |
| 7 | Investigations Closure | 9.0% | 5 | 0.45 |
| 8 | Stability OTIF | 9.0% | 4 | 0.36 |
| 9 | Change Control Closure | 9.0% | 4 | 0.36 |
| 10 | Deviation Closure | 9.0% | 4 | 0.36 |
| eleven | SOP Validity | 3.0% | 4 | 0.12 |

### Example 2

Implementation of Quality Health Barometer at Site B:

The baseline score of “Site B” prior to implementation of Quality Health Barometer was determined to be 3.5 which means the overall site health is considered to have “Acute Infrequent symptoms of quality issue” and needs improvement.

Using the Quality Health Barometer, eleven critical indicators of quality were evaluated for Site B.

The score is tabulated in Table 5:

TABLE 5
“SITE B” - Baseline score

| | | | | Overall Score |
|---|---|---|---|---|
| | | | | 3.5 |
| | | | | Overall |
| | | | | Site Health |
| | | | | Needs |
| | | Weight- | | improvement |
| Sr. No. | Indicator | age | Rating | Score |
| 1 | Audit Score card | 22.0% | 4 | 0.88 |
| 2 | Data Integrity Compliance | 9.0% | 4 | 0.36 |
| 3 | Complaint rate | 9.0% | 5 | 0.45 |
| 4 | Invalidated OOS | 9.0% | 4 | 0.36 |
| 5 | CAPA Closure rate | 9.0% | 5 | 0.45 |
| 6 | Process OOS | 3.0% | 5 | 0.15 |
| 7 | Investigations Closure | 9.0% | 4 | 0.36 |
| 8 | Stability OTIF | 9.0% | 1 | 0.09 |
| 9 | Change Control Closure | 9.0% | 1 | 0.09 |
| 10 | Deviation Closure | 9.0% | 3 | 0.27 |
| eleven | SOP Validity | 3.0% | 1 | 0.03 |

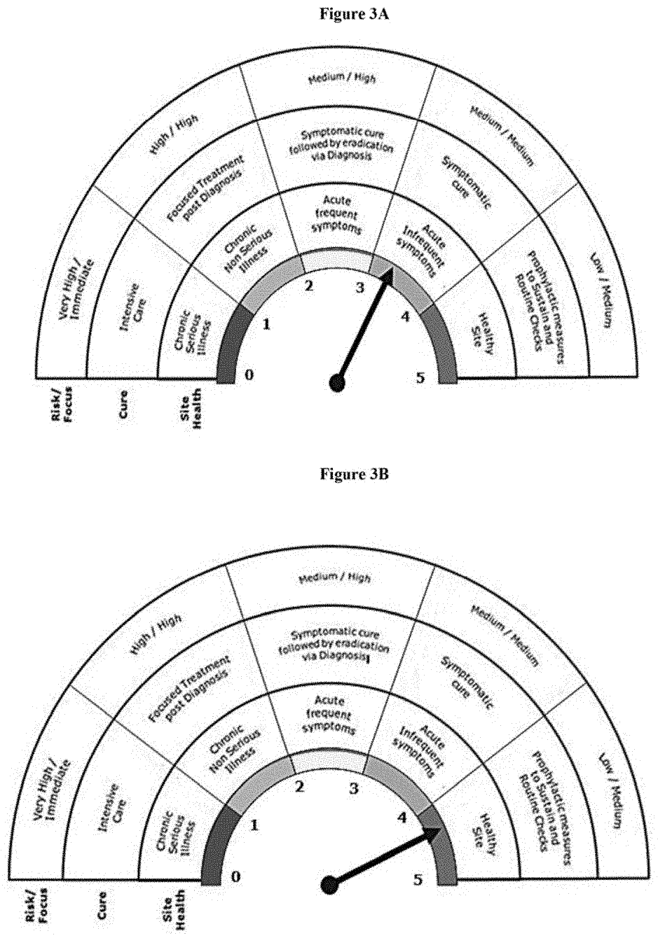

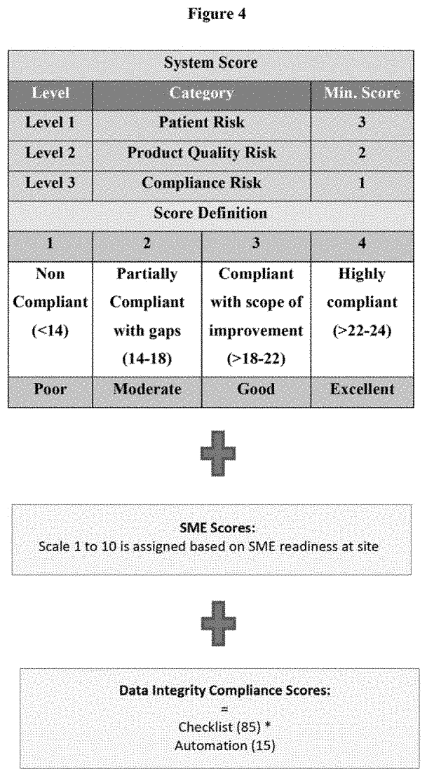

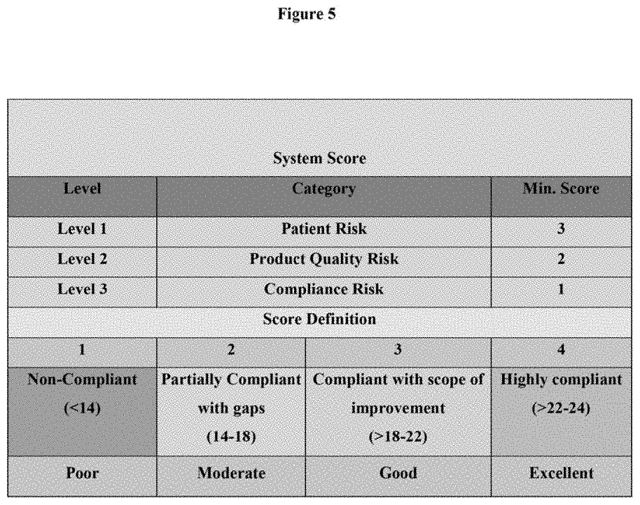

From Table 5 it was observed that, there is a need to improve the score on many of the indicators namely Stability OTIF, Change control closure, SOP Validity as depicted in FIG. 3A to improve the health of “Site B”. Accordingly, measures were taken to improvise on all the critical indicators with continuous monitoring of sustenance of other indicators so as to bring the site score to 4.1 which terms Site B as a “Healthy Site”. The improvised scores are depicted in table 6. In other words, the risk at said site is now considered to be Low and the focus on quality issue is Medium with Prophylactic measures to sustain routine checks. The result on the quality health barometer is depicted in FIG. 3B.

TABLE 6
“SITE B” - Improvised score

| | | | | Overall Score |
|---|---|---|---|---|
| | | | | 4.1 |
| | | | | Overall |
| | | | | Site Health |
| | | Weight- | | Very Healthy |
| Sr. No. | Indicator | age | Rating | Score |
| 1 | Audit Score card | 22.0% | 4 | 0.88 |
| 2 | Data Integrity Compliance | 9.0% | 4 | 0.36 |
| 3 | Complaint rate | 9.0% | 5 | 0.45 |
| 4 | Invalidated OOS | 9.0% | 4 | 0.36 |
| 5 | CAPA Closure rate | 9.0% | 5 | 0.45 |
| 6 | Process OOS | 3.0% | 5 | 0.15 |
| 7 | Investigations Closure | 9.0% | 4 | 0.36 |
| 8 | Stability OTIF | 9.0% | 4 | 0.36 |
| 9 | Change Control Closure | 9.0% | 4 | 0.36 |
| 10 | Deviation Closure | 9.0% | 3 | 0.27 |
| eleven | SOP Validity | 3.0% | 4 | 0.12 |

### Example 3

The score of site “A” for Quality tools are on lower side as per table 7, where audit score observed is poor and site is non-compliant against the six system requirement. Also few SME are found less competent during audit facing but can be trained and DI score observed is on lower side as below:—

TABLE 7

| Quality Tool | Scores of Site A |
|---|---|
| Audit Score | 14 |
| DI Score | 51 |
| SME Score | 5 |

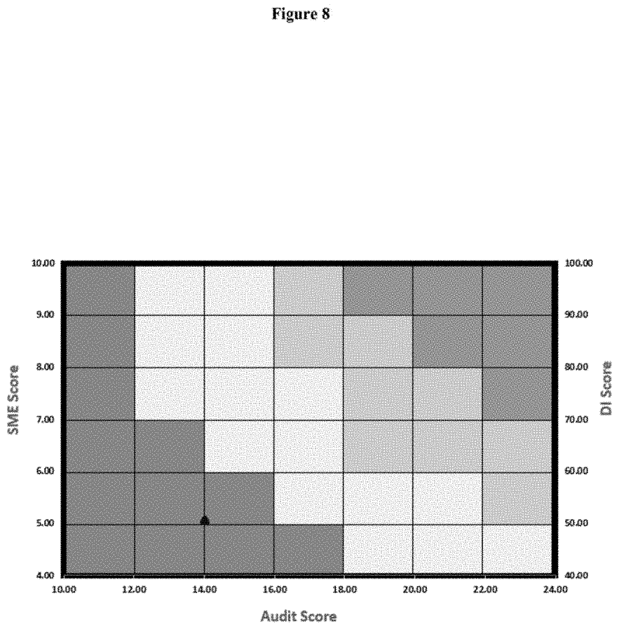

The result is depicted in FIG. 8.

Predict Interpretation of Site a Based on FIG. 8:

Based on the computation of above scores of quality tool, the outcome of site A for regulatory audit is predicted poor (Red block) and with double digit WL/483 can be interpreted. This weak condition of site A can be improved by mainly focusing on improvement in six system compliance, training the SME in relevant subject and by increasing compliance of DI score.

### Example 4

The score of site “A” for Quality tools are improvised as per table 8, where audit score observed is moderate and site is partially compliant with gaps against the six system requirement. Also SME (mainly Quality) require extensive level of audit facing training while possessing requisite knowledge and DI score observed at moderate side as below:—

TABLE 8

| Quality Tool | Scores of Site A |
|---|---|
| Audit Score | 16 |
| DI Score | 65 |
| SME Score | 6 |

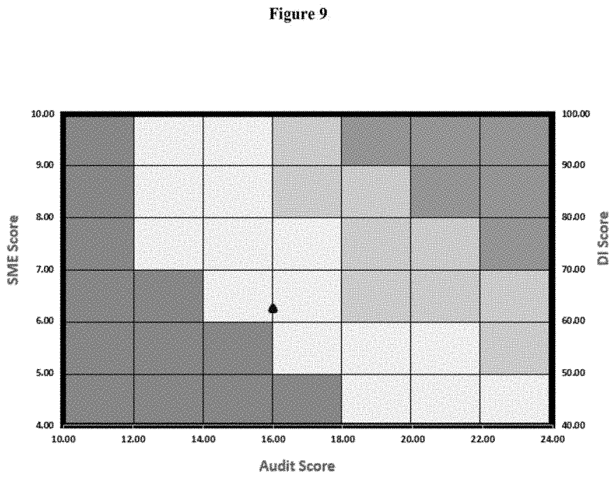

The result is depicted in FIG. 9.

Predict Interpretation of Site A Based on FIG. 9:

Based on the computation of above scores, the outcome for site A for regulatory audit is predicted moderate (Yellow block) and less than seven 483 can be interpreted with no critical observation. This can be improved by mainly focusing on improvement in six system compliance, training the SME in respective subject and by increasing compliance of DI score.

### Example 5

The score of site “A” for Quality tools are with some improvements as per table 9, where audit score is observed to be good and site is compliant with scope of improvement against the six system requirement. Also SME (mainly but not only quality) require low level of audit facing training and DI score observed at above qualifying level as below:—

TABLE 9

| Quality Tool | Scores of Site A |
|---|---|
| Audit Score | 19 |
| DI Score | 75 |
| SME Score | 8 |

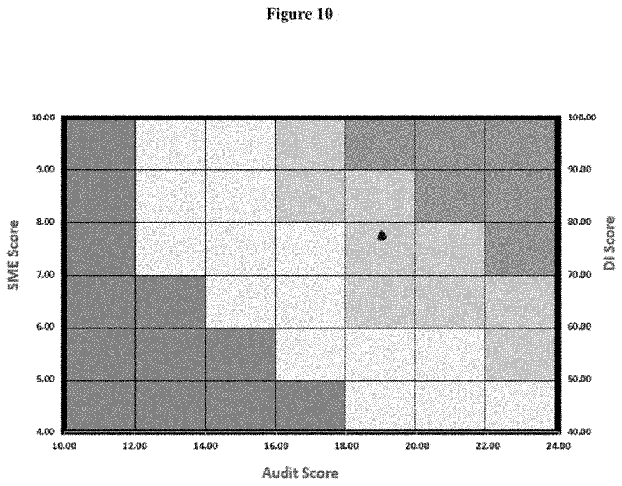

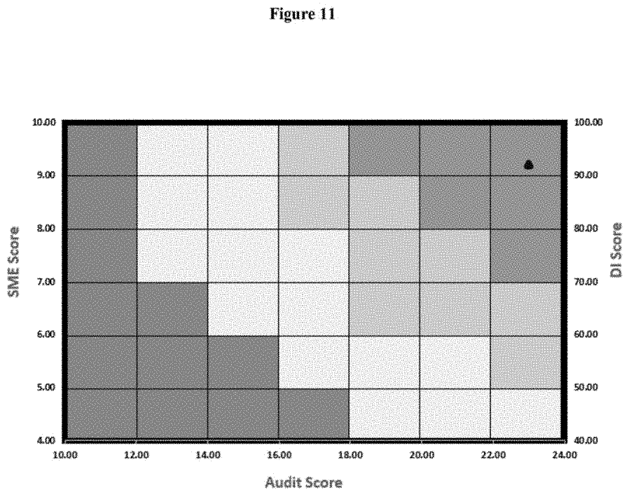

The result is depicted in FIG. 10.

Predict Interpretation of Site A Based on FIG. 10:

Based on the computation of above scores, the outcome for site A for regulatory audit is predicted good (light green) and less than three 483 can be interpreted with no critical and few major observation. This can be improved by mainly focusing on improvement in six system compliance, training the SME in audit facing and by increasing compliance of DI score.

### Example 6

The score of site “A” for Quality tools are with some improvements as per table 10, where audit score observed is excellent and site is highly compliant against the six system requirement. Also SME (mainly quality) requires only incidence related direction or guidance during audit to defend our case as required by regulation during inspection when required and DI score observed at good complaint level as below:—

TABLE 10

| Quality Tool | Scores of Site A |
|---|---|
| Audit Score | 23 |
| DI Score | 94 |
| SME Score | 9 |

The result is depicted in FIG. 11.

Predict Interpretation of Site A Based on FIG. 11:

Based on the computation of above scores, the outcome for site A for regulatory audit is predicted excellent (dark green) and less than two 483 can be interpreted with no critical and no major observation.

### Example 7

The score of site “A” for Data Integrity compliance is depicted in table 11, where the scores from DI checklist and the automation are on the lower side. The site is having inadequate data governance, data retention, no audit trail, lack of DI training and undefined review and approval procedures. The site is also lacking in automation in manufacturing, quality control and quality assurance systems.

TABLE 11

| | % Contribution | | Total DI |
|---|---|---|---|
| | factors used for | Scores of | Score of |
| Parameters | computation | Site A | Site A |
| DI checklist score | 85 | 44.3489 | 48.34 |
| Automation score | 15 | 4 | |

CALCULUS™ Interpretation at Site A:

Based on the computation of above scores, the site “A” is rated as C for compliance to data integrity. This weak condition of site A can be improved by mainly focusing on improvements in record management, data governance, data review and approval, trainings and in automation, which will result in increase of DI compliance score.

### Example 8

The score of site “A” for CALCULUS™ are depicted in table 12, where although the automation score is low but there are improvements in the DI checklist scores. The site has initiated the training programs on data integrity, which is leading to awareness amongst the employees and in turn making them to exercise appropriate methods/controls during data review and approval.

TABLE 12

| | | Scores of | Total DI |
|---|---|---|---|
| Parameters | % Contribution | Site A | Score of Site A |
| DI checklist score | 85 | 58.1725 | 62.7 |
| Automation score | 15 | 4.0 | |

CALCULUS™ Interpretation at Site A:

Based on the computation of above scores, the site “A” is rated as B for compliance to data integrity. This condition of site A can be improved further by focusing more on improvement in recording and collection of data, data governance and in automation, which will result in increase of DI compliance score.

### Example 9

The score of site “A” for CALCULUS™ are depicted in table 13, and it can be seen that there are improvements in the DI checklist score and automation score. The site has built a data governance mechanism and procedures for handling data integrity incidents. The site has also initiated the data integrity audits at sites and reviews with leadership team to have continuous vigilance. In terms of automation, site has improvised the computerized systems transactions, activated the audit trails for all the computerized systems and automated some of the Quality Control and production functions. The site has also prepared a risk assessment report for all the standalone instruments in use at site.

TABLE 13

| | | Scores of | Total DI |
|---|---|---|---|
| Parameters | % Contribution | Site A | Score of Site A |
| DI checklist score | 85 | 79.5090 | 86.51 |
| Automation score | 15 | 7 | |

CALCULUS™ Interpretation at Site A:

Based on the computation of above scores, the site “A” is rated as A for compliance to data integrity. The condition of site A can be improved further by focusing more on the importance of validation of computerized systems and increasing the automation at production, quality assurance and control functions.

### Example 10

The improvised scores of site “A” for CALCULUS™ are depicted in table 14. The site has improvised the data integrity compliance in the systems and has increased the automation of the functions of Quality Control, Quality Assurance and production like modules of document management, Quality Management Systems and training management.

TABLE 14

| | | Scores | Total DI |
|---|---|---|---|
| Parameters | % Contribution | of Site A | Score of Site A |
| DI checklist score | 85 | 84.2185 | 94.21 |
| Automation score | 15 | 10 | |

CALCULUS™ Interpretation at Site A:

Based on the computation of above scores, the site “A” is rated as A+ for compliance to data integrity. The above score can further be improved by maintaining consistent compliance in the processes with respect to data integrity, data governance and by increasing the automated functions in Quality Control, Quality Assurance and production.

We claim:

1. A level meter measuring the quality health of a pharmaceutical manufacturing site and which predicts 24/7 audit readiness for quality outcomes, wherein outcome is interpreted using tangible data; referred to as a Quality Health Barometer (SENSOR™).

2. The level meter as claimed in claim 1, wherein the score on the level meter is the site score for a pharmaceutical manufacturing site under evaluation, which is determined using eleven critical indicators selected from the group consisting of:
1. Audit score card (System Score),
2. Data integrity compliance score,
3. Product Quality Complaints,
4. Invalidated OOS,
5. CAPA closure rate,
6. Process OOS,
7. Investigation Closure Rate,
8. Stability OTIF,
9. Change Control Closure rate,
10. Deviation closure rate and
11. SOP Validity.

3. The level meter as claimed in claim 2, wherein each of the eleven critical indicators is attributed a weightage in percent based on the influence of the indicator on quality, selected from:
(a) Audit score card (System score) with a weightage of 22%;
(b) Data integrity compliance, complaints, Invalidated OOS, CAPA closure rate, Investigation closure rate, Stability OTIF, Change control closure rate & Deviation closure rate with a weightage of 9% each and
(c) Process OOS and SOP validity with a weightage of 3% each.

4. The level meter as claimed in claim 2, wherein each of the eleven critical indicators is allocated a rating between 1 to 5.

5. A method to evaluate the site score as claimed in claim 2, comprising the steps of:
(a) providing audit score card (System score) with a weightage of 22%;
(b) providing data integrity compliance, complaints, Invalidated OOS, CAPA closure rate, Investigation closure rate, Stability OTIF, Change control closure rate & Deviation closure rate with a weightage of 9% each;
(c) providing process OOS and SOP validity with a weightage of 3% each;
(d) providing ratings on scale of 1 to 5 based on threshold value and targets for improvements assigned, and their current status, wherein:
‘5’ being considered as 100% achievement over the target assigned and
‘1’ being considered as achievement way below minimum expected level of target;
(e) obtaining the score for each of the eleven critical indicators as a product of weightage of steps (a) to (c) and the rating of step (d);
(f) obtaining the site score by adding the scores of each of the eleven critical indicators of step (e) and
(g) mapping the site score obtained in step (f) onto the level meter with a pointer configured by application of software to indicate a region on the level meter in order to gauge the performance of the site.

6. The level meter as claimed in claim 1, wherein the meter is demarcated into 5 colored regions corresponding to the score and indicative of ‘Site Health’, selected from:
a) Score of 0-1 corresponding to red region indicating that the Site Health has Chronic Serious Illness,
b) Score of 1-2 corresponding to orange region indicating that the Site Health has Chronic Non-serious Illness,
c) Score of 2-3 corresponding to yellow region indicating that the Site Health has Acute Frequent Symptoms,
d) Score of 3-4 corresponding to light green region indicating that the Site Health has Acute In-frequent Symptoms, and
e) Score of 4-5 corresponding to green region indicating that the Site is Healthy.

7. The level meter as claimed in claim 1, wherein the meter is demarcated into 5 colored regions corresponding to the score and indicative of ‘Cure’, selected from:
a) Score of 0-1 corresponding to red region indicating that the Cure required is Intensive Care,
b) Score of 1-2 corresponding to orange region indicating that the Cure required is Focused Treatment post Diagnosis,
c) Score of 2-3 corresponding to yellow region indicating that the Cure required is Symptomatic cure followed by eradication via Diagnosis,
d) Score of 3-4 corresponding to light green region indicating that the Cure required is Symptomatic cure, and
e) Score of 4-5 corresponding to green region indicating that the Cure required is Prophylactic measures to Sustain and Routine Checks.

8. The level meter as claimed in claim 1, wherein the meter is demarcated into 5 colored regions corresponding to the score and indicative of ‘Risks’ and ‘Focus’, selected from:
a) Score of 0-1 corresponding to red region indicating that the Risk is Very High and Focus is Immediate,
b) Score of 1-2 corresponding to orange region indicating that the Risk is High and Focus is High,
c) Score of 2-3 corresponding to yellow region indicating that the Risk is Medium and Focus is High,
d) Score of 3-4 corresponding to light green region indicating that the Risk is Medium and Focus is Medium, and
e) Score of 4-5 corresponding to green region indicating that the Risk is Low and Focus is Medium.

9. A quality tool (PREDICT™) used for predicting audit or inspection outcome at pharmaceutical manufacturing sites, wherein measurement is based on intangible data and the outcome is useful to interpret the probable outcome of regulatory inspections at a site.

10. A method of evaluation (PREDICT™) of the predict system score of a pharmaceutical manufacturing site, wherein the predicted outcome comprises of three elements:
(i) first element or audit score (System score) being assessment of GMP system compliance via check points based on 132 vital parameters;
(ii) second element being SME readiness, which integrates aspects of SME readiness at sites; and
(iii) third element being data integrity compliance scores.

11. The method as claimed in claim 10, wherein the 132 parameters of the first element are categorized into three levels namely Patient Risk (Level-I), Product Quality Risk (Level-II) and Compliance Risk (Level-III).

12. The method as claimed in claim 10, wherein the first element is assessed according to the steps comprising of:
(i) associating each of the 132 parameters with a weightage (%) based on its relevance to patient (highest weightage), product and compliance;
(ii) providing a rating to each of the parameters;
(iii) obtaining the audit score for each of the parameters as a product of weightage and the rating; and
(iv) obtaining the final audit score on a scale of 1 to 24 as an average of the individual scores wherein:
a) Scores ranging from above 22-24 indicates that the site is highly compliant.
b) Scores ranging from above 18-22 indicates that the site is compliant with scope of improvement;
c) Scores ranging from 14-18 indicates that the site is partially compliant with gaps; and
d) Scores less than 14 indicates that the site is non complaint.

13. The method as claimed in claim 12, wherein in step (ii), each of the 132 parameters are assigned with ratings on a scale of 0 to 4 based on level of compliance, wherein:
a) 0 signifies “No process available”,
b) 1 signifies “Non-compliant process”,
c) 2 signifies “Partially compliant process”,
d) 3 signifies “Compliant with improvements” and,
e) 4 signifies “Highly compliant process”.

14. The method as claimed in claim 10, wherein the second element or the SME readiness is provided a score selected from:
a) “0” Need major change in SME at all levels including quality.
b) “1” Few critical SMEs (audit facing) in quality requires replacement.
c) “2” Few critical SME (audit facing) within other 5 systems requires replacement.
d) “3” Challenge only with 1 or 2 SME (audit facing) which can be bridged by other competent staff under or at Peer level.
e) “4” Gap in Quality SME (audit facing) but can be trained.
f) “5” Gap in few other SMEs (audit facing) but can be trained.
g) “6” SME (mainly Quality) require extensive level of audit facing training while possessing requisite knowledge.
h) “7” SME (mainly but not only quality) require major level of strategic audit facing training.
i) “8” SME (mainly but not only quality) require low level of audit facing training.
j) “9” SME (mainly quality) requires only incidence related direction or guidance during audit to defend our case as required by regulation during inspection when required.
k) “10” SME (mainly quality) is self-sufficient in changing the course of inspection in our favour due to strong regulatory knowledge and negotiation capability.

15. The method as claimed in claim 10, wherein the third element or data integrity compliance is provided a score in percentage (%) based on the compliance level at the site.

16. A quality tool (CALCULUS™) used for evaluating the data integrity compliance at pharmaceutical manufacturing site, wherein measurement is based on quantified data.

17. A method of evaluation (CALCULUS™) of the data integrity compliance at a pharmaceutical manufacturing site which is determined based on regulatory requirements and also internal need for increasing and elevating automation to reduce possibility of errors.

18. The method according to claim 17, wherein the data integrity compliance is provided a score (DI score) computed by considering the sum of a weightage of 85% for a first component including a data integrity checklist score and 15% for a second component including level of automation at site; wherein the pharmaceutical manufacturing site is provided a rating of A+, A, B or C based on the DI score.

19. The method according to claim 18, wherein the first component for measuring the data integrity compliance is based on data integrity checklist consisting of 163 parameters for assessing compliance level of sites with regards to regulatory requirements.

20. The method according to claim 18, wherein the 163 parameters are selected from:
a) Recording and collection of data
b) Original record/True copy
c) Excluding data,
d) Data Processing
e) Data transfer/mitigation
f) Data Governance
g) Data Integrity Risk Assessment (DIRA)
h) Computerized System transactions
i) Computerized system User access/System Administrator role
j) Audit Trail
k) Electronic Signatures
l) Data review and approval
m) Data Retention
n) Back up and Archive
o) File structure
p) Validation
q) IT supplier and Service provider
r) Quality Management Systems (QMS)
s) Calibration
t) Quality Control
u) Standalone Systems
v) Trainings
w) Manufacturing

21. The method according to claim 18, wherein the second component for measuring the data integrity compliance is based on the level of automation at site comprising weightage of 5% for Quality Control, 5% for Quality Assurance and 5% for Manufacturing.

22. The method according to claim 17, wherein the data integrity compliance at pharmaceutical manufacturing site requires a threshold value of >70%.

23. The method according to claim 18, wherein:
(i) the rating of A+ is assigned to a site with DI score of >90%;
(ii) the rating of A is assigned to a site with DI score of >70-90%;
(iii) the rating of B is assigned to a site with DI score of 50-70%;
(iv) the rating of C is assigned to a site with DI score of <50%; and
(v) the qualifying rating for the site as data integrity compliant is A+ or A.
